(19) United States
(12) Patent Application Publication
(10) Pub. No.: US 2023/0037570 A1
(43) Pub. Date: Feb. 9, 2023

(54) Distal head for an endoscope, said distal head being provided with an enlarged working channel

(71) Applicant: AXESS VISION TECHNOLOGY, JOUE-LES-TOURS (FR)

(72) Inventors: Emmanuel HALLAUER, SACHE (FR); Philippe LE ROUX, TOURS (FR)

(21) Appl. No.: 17/793,063

(22) PCT Filed: Dec. 22, 2020
(86) PCT No.: PCT/FR2020/052594

§ 371 Date: Jul. 15, 2022

(30) Foreign Application Priority Data
Jan. 17, 2020 (FR) FR2000444

(51) Int. Cl. A61B 1/015; A61B 1/00; A61B 1/018

(52) U.S. Cl. CPC A61B 1/015; A61B 1/00094; A61B 1/018; A61B 1/00096; A61B 1/005

(57) ABSTRACT

The disclosure relates to a distal endoscope head including a support for a vision system, the support being delimited on one side, by a proximal face and, on the opposite side, by a distal face into which the vision system opens out, the support being traversed by at least one hole of an operating channel opening out into the distal face by an ovoid section which decreases from the distal face of the support in the direction of the proximal face of the support.

### TECHNICAL FIELD

The present disclosure relates to the technical field of medical endoscopes in a general sense for accessing the interior of a body such as a cavity or a channel for example and it relates to reusable or single-use endoscopes.

The present disclosure more specifically relates to the distal head of such endoscopes, adapted to ensure multiple functions such as the visualization, the supply of fluid, the aspiration of fluid, the supply of instruments, the collection of samples or the conduction of surgical procedures for example.

The endoscope equipped with the distal head in accordance with the disclosure finds particularly advantageous applications for allowing access to the inner surface of a hollow organ, of a cavity or of a natural or artificial conduit of the human body with a view to performing various operations for therapeutic, surgical or diagnostic purposes.

The endoscope according to the disclosure is used for diagnostic, therapeutic or surgical purposes for the inspection of all inner parts of the human body accessible by the natural or artificial path. For example, the endoscope according to the disclosure can be used in the field of urinary tract, gastrointestinal tract, respiratory system, cardiovascular system, trachea, the sinus cavity, women's reproductive system, abdominal cavity or any other part of the human body to be explored by a natural or artificial path.

### BACKGROUND

In general, a medical endoscope includes, as described for example by patent application WO 2014/106510, a control handle to which a tubular structure is fixed including a distal head equipped with a vision system that allows illuminating and examining the organ, the cavity or the conduit of the human body. Upstream of the distal head, the tubular insertion structure includes a deflection part that allows orienting the distal head inside the insertion path.

This tubular structure is adapted to allow bringing at the distal head, one or several devices designed to perform different functions such as for example the supply of fluid, the aspiration of fluid, the supply of instruments, the collection of samples or the conduction of surgical procedures. The distal head of the endoscope is also equipped with at least one channel called operating or working channel to allow the passage of these various devices.

Thus, such an endoscope is provided with a multifunction distal head configured to allow the implantation of the vision system and the passage of these various devices. However, this distal head has a passage or bulk section which is limited by the smallest width of the natural or artificial access path in which the tubular structure is engaged. Given the diversity of the functionalities that an endoscope must have and the dimensional constraints imposed by the access path, there is a difficulty in configuring the distal head for some functions of the endoscope to be carried out. This is in particular the case with the function of aspiration of liquids, the removal of bulky bodies or the use of cumbersome tools such as biopsy forceps. Suction phenomena on the mucous membranes which may lead to hemorrhages also appear. In addition to the resulting performance losses, the distal head of such an endoscope is relatively complex to produce, increasing its manufacturing cost.

Patent application US 2017/0143199 describes a bronchoscope including a rigid tube delimiting a working channel separated from a hardware channel by an inner wall. A lighting component and an imaging component are fixed to the distal end of the hardware channel. The rigid tube is beveled at the distal end to increase the size of the opening of the working channel and reduce the overall dimension of the distal end of the tube. If the distal end of such a tube is reduced, its beveled shape does not offer total security for accessing the interior of bodies having access paths with particular shapes. Such a shape is moreover less effective in ensuring good aspiration of the liquids, without pressure drop.

Patent application EP 1 772 095 describes an endoscope provided with an instrument adapted to pivot at its distal end. The instrument is inserted into the operating channel by opening out through an elliptical-shaped opening, in a housing arranged in the distal head and in which a pivotally actuated support is housed. This endoscope has a distal head with a significant bulk and is in particular not suitable for fluid aspiration.

Patent application US 2013/0150667 describes an endoscope having in particular an operating channel including a conductive tubular insert mounted on one side in a housing arranged in the distal head and on the other side, in a tube. There is no change in the section of said operating channel that allows increasing the aspiration, at the inlet on the distal side. This endoscope has a distal head with a significant bulk.

### SUMMARY

The present disclosure therefore aims to overcome the drawbacks of the state of the art by proposing a new distal endoscope head of simple and inexpensive design, adapted to allow the complete and effective implementation of the different functions of the endoscope.

The present disclosure also aims to propose a new distal endoscope head offering a limited bulk while allowing optimizing the aspiration of the fluids and limiting the suction phenomena.

To achieve such an objective, the distal endoscope head includes a support for a vision system, the support being delimited on one side, by a proximal face and, on the opposite side, by a distal face parallel to the proximal face and into which the vision system opens out, the support being traversed by at least one hole of an operating channel opening out into the distal face by an ovoid section whose projected surface with respect to the axis of the hole decreases from the distal face of the support in the direction of the proximal face of the support, the ovoid section of the operating channel in the distal face occupying a surface greater than or equal to at least 50% of the surface of the distal face of the support.

Typically, the ovoid section of the hole of the operating channel in the distal face of the support occupies a surface comprised between 50% and 60% of the area of the distal face of the support.

For example, the ovoid section of the hole of the operating channel evolves from the distal face of the support, ending with a circular section.

Advantageously, the ovoid section of the hole of the operating channel evolves from the distal face of the support, ending with a circular section, at an abutment surface for one end of the conduit delimiting the operating channel.

According to one preferred exemplary embodiment, the ovoid section of the hole of the operating channel is delimited at the distal face, by a peripheral circular arc-shaped edge concentric with the outer surface of the support and connected on either side, by connecting edges, to a central circular arc-shaped edge whose radius is greater than the radius of the peripheral edge.

According to one advantageous alternative embodiment, the support is in the form of a ring delimited on one side by the distal face and on the other side by the proximal face, the distal and proximal faces being parallel to each other and perpendicular to the longitudinal axis of the ring.

According to one advantageous alternative embodiment, the hole of the operating channel is delimited by an inner surface provided, from the distal face of the support, with helically-shaped grooves or ribs.

Advantageously, the support includes a passage for mounting, as a video system, an optical lens or a vision sensor and at least one light source, coated along the distal face of the support with a transparent protective material.

According to one exemplary embodiment, the optical lens or the vision sensor and at least the light source are embedded into the mounting passage, by the transparent protective material.

For example, the support includes, from the proximal face, a cylindrical section extended by a narrowed section decreasing up to the distal face of the support to present at the distal face, a pseudo-triangular section.

In practice, the passage opens out into the part of the distal face, located opposite to the semi-circular edge, between the central edge of the support and the central edge delimiting the hole.

Another object of the disclosure is to propose an insertion tube connected, at its proximal part, to a control handle and equipped, at its distal part, with a distal head in accordance with the disclosure.

Various other characteristics emerge from the description given below with reference to the appended drawings which show, by way of non-limiting examples, embodiments of the object of the disclosure.

### BRIEF DESCRIPTION OF THE DRAWINGS

FIG. 1 is a general view of an endoscope in a general sense equipped with a distal head in accordance with the disclosure.

FIG. 2 is a view showing more particularly the deflection part of the distal head in accordance with the disclosure.

FIG. 3 is a perspective of the distal head in accordance with the disclosure seen from its distal face.

FIG. 4 is a perspective of the distal head seen from its proximal face.

FIG. 5 is a plan view of the distal head taken from its distal face.

FIG. 6 is a plan view of the distal head taken from its proximal face.

FIG. 7 is a view similar to FIG. 4 showing the distal head equipped with the operating conduit.

FIG. 8 is a similar view of FIG. 3, showing the distal head equipped with the operating conduit.

FIG. 9 is a front view of another exemplary embodiment of the distal head in accordance with the disclosure.

### DETAILED DESCRIPTION

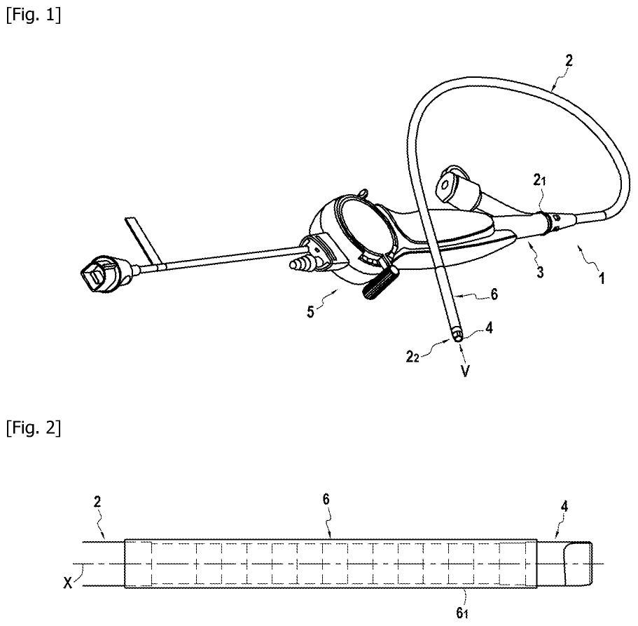

FIG. 1 illustrates by way of example, a medical endoscope 1 in a general sense designed to access the interior of a body such as a cavity or a channel for example. Conventionally, an endoscope 1 includes an insertion tube 2 having, on one side, a proximal part 2₁ connected to a control handle 3 and, on the opposite side, a distal part 2₂ equipped with a distal head 4 in accordance with the disclosure. The insertion tube 2 is fixed temporarily or permanently on the control handle 3. This insertion tube 2 which has a more or less significant length and a flexibility is intended to be introduced into a natural or artificial access path with a view to performing various operations or functions for therapeutic, surgical or diagnostic purposes. The insertion tube 2 is made of a semi-rigid material and has a length adapted to the length of the conduit to be inspected and which can be comprised between 5 cm and 2 m. The insertion tube 2 has various cross-sectional shapes such as square, oval or circular shapes. This insertion tube 2 which is in contact with the tissues, the human organs or medical devices (trocars or probes), is essentially for single or multiple use for a patient or is even reusable after decontamination, disinfection or sterilization.

Conventionally, the endoscope 1 in accordance with the disclosure includes a vision system V able to illuminate and bring back an image of the distal part of the insertion tube 2. The endoscope 1 thus includes a vision system mounted inside the control handle 3 and entering inside the insertion tube 2 up to the distal head 4.

Conventionally, the endoscope 1 also includes a control mechanism 5 that allows orienting the distal head 4 with respect to the longitudinal axis X of the insertion tube 2. For this purpose, the insertion tube 2 includes, upstream of the distal head 4, a bending, folding or deflection part 6 allowing the orientation of the distal head 4 with respect to the longitudinal axis X of the insertion tube 2 (FIG. 2).

The control mechanism 5 can be made in any suitable manner such that the distal head 4 can be moved between a rest position in which the insertion tube 2 is rectilinear and a deflected position in which the deflection part 6 is curved. By way of non-limiting example, the control mechanism 5 can correspond to the control mechanism described in patent FR 3 047 887. For this purpose, the control mechanism 5 includes a manual control lever driving in rotation a pulley on which, in order to be fixed to the distal head 4, at least one cable mounted inside the insertion tube 2 is fixed.

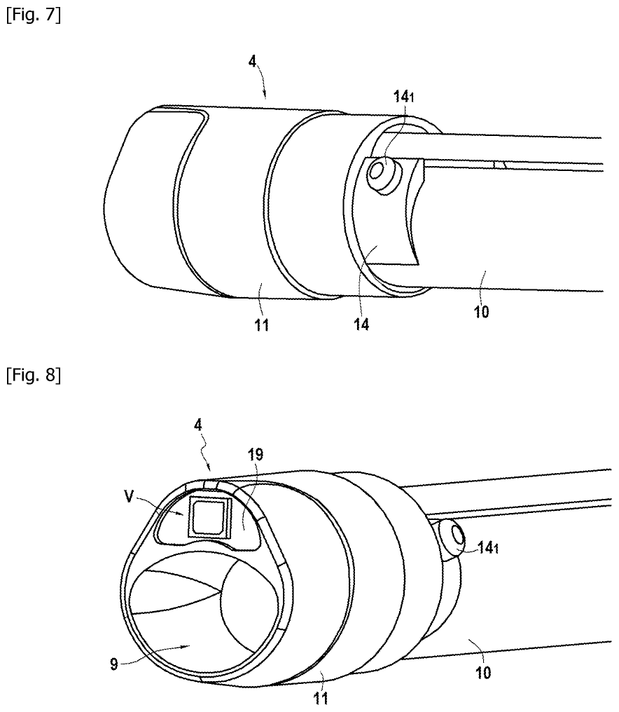

Conventionally, the endoscope 1 also includes an operating or working channel 9 extending from the control handle 3 to the distal head 4 to allow the supply of various tools and/or fluids and/or the aspiration of fluids. This operating channel 9 is delimited by a pipe or conduit 10 positioned inside the insertion tube 2 (FIGS. 7, 8).

In accordance with the disclosure, the distal head 4 includes a body or a support 11 in the form of a ring delimited on the side closest to the control handle 3, by a proximal face 11p and on the opposite side farthest from the control handle, by a distal face 11d into which the vision system V opens out. As shown in the drawings, this ring or support 11 is delimited on one side by the distal face 11d and on the other side by the proximal face 11p. The distal face 11d and the proximal face 11p are parallel to each other and perpendicular to the longitudinal axis of the ring or of the support.

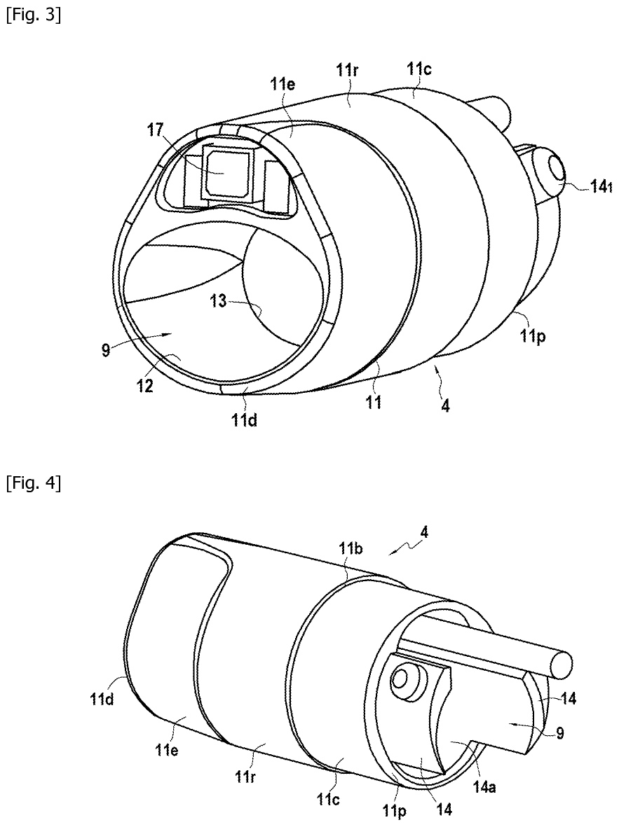

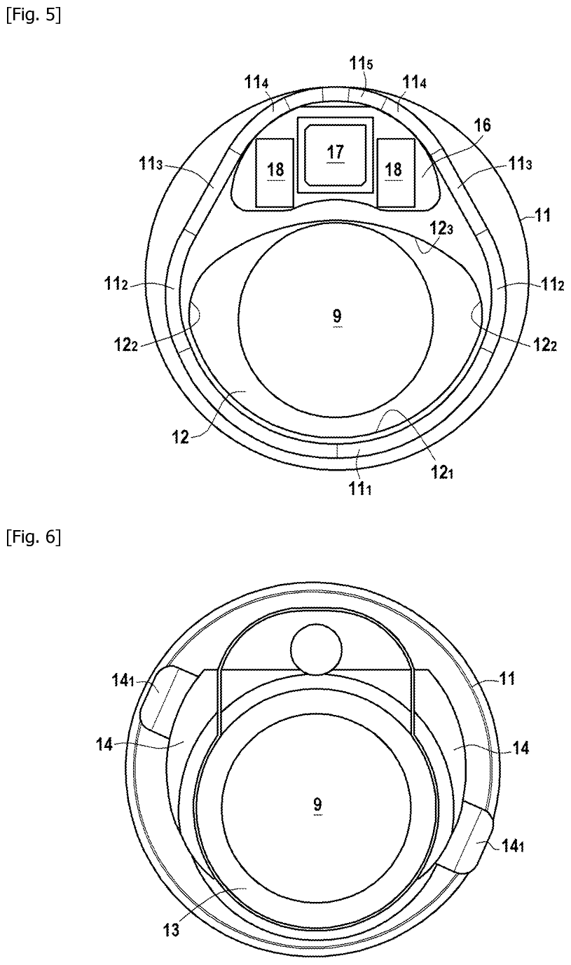

As shown in FIGS. 3 and 4, the support 11 includes, from the proximal face 11p, an outer surface delimiting a circular cylindrical section 11c extended by a narrowed section 11e decreasing up to the distal face 11d of the support 11. In the exemplary embodiment illustrated in FIGS. 3, 5 and 8, the distal face 11d of the support 11 has a section or a pseudo-triangular profile delimited by a semi-circular edge 11₁ extending on either side, by connecting edges 11₂, to converging rectilinear edges 11₃ connecting, by connecting edges 11₄, to a central circular arc-shaped edge 11₅, of a radius smaller than the radius of the semi-circular edge 11₁. The narrowed section 11e of the support thus has a profile which evolves from a pseudo-triangular profile to a circular profile connecting, by a cylindrical connecting section 11r, to the circular cylindrical section 11c of the support 11. The circular cylindrical section 11c has a diameter smaller than the diameter of the cylindrical connecting section 11r to define a shoulder 11b. This circular cylindrical section 11c is adapted to receive a protective sheath 6₁ for the deflection part 6.

The support 11 of the distal head 4 is traversed right through, by the operating channel 9 to open out into the distal face 11d. In the exemplary embodiment illustrated in the drawings (FIGS. 3 to 6), a hole 12 is arranged in the support 11 from the distal face 11d to form the terminal part of the operating channel 9. This hole 12 extends up to an abutment surface 13 arranged in the support 11 to serve as an abutment at the end of the conduit 10 which thus communicates with this hole 12.

As shown more specifically in FIGS. 4 and 7, the support 11 has two fixing arms 14 protruding from the proximal face 11p of the support, to delimit therebetween a passage 14a for the conduit 10 which is bearing on the abutment surface 13. These two fixing arms 14 thus act as fixing clamps for the conduit 10 so that the operating channel 9 thus extends from the control handle 3 up to the distal face 11d of the distal head 4, using the conduit 10 extending through the hole 12. Thus, the conduit 10 is engaged from the proximal face 11p of the support, inside a housing of the support 11 to bear on the abutment surface 13 located set back from the proximal face 11p of the support. For example, each fixing arm 14 includes a stud 14₁ intended to cooperate with an oblong opening arranged in the deflection part 6 to constitute a bayonet assembly between the distal head 4 and the deflection part 6.

According to an aspect of the disclosure, the operating channel 9 has at the distal head 4, a section which decreases in the direction of the proximal face 11p of the support 11. In the exemplary embodiment illustrated in the drawings, the hole 12 of the support 11 has a section which decreases in the direction of the proximal face 11p of the support 11. The section of the hole 12 is thus the smallest at the abutment surface 13 and widens in the direction of the distal face 11d of the support 11.

The operating channel 9 has a section which decreases from the distal face 11d of the support 11. In the exemplary embodiment illustrated in the drawings, the hole 12 has a section which decreases from the distal face 11d of the support and up to the abutment surface 13. The section of the hole 12 is thus the smallest at the abutment surface 13 and widens up to the distal face 11d of the support 11. For example, the section of the hole 12 gradually decreases between the distal face 11d of the support 11 and the abutment surface 13.

According to another aspect of the disclosure, the operating channel 9 opens out into the distal face 11d of the support 11 by an ovoid section. In the illustrated exemplary embodiment, the hole 12 is delimited at the distal face 11d, by a peripheral circular arc-shaped edge 12₁ connected on either side, by connecting edges 12₂, to a central circular arc-shaped edge 12₃ whose radius is greater than the radius of the peripheral edge 12₁. For example, the peripheral edge 12₁ is concentric with the support 11 and more specifically with the outer surface of the support 11 (narrowed section 11e) to maximize the aspiration area, and has a radius of curvature R₁. Advantageously, the central edge 12₃ has a radius of curvature, R₂, such that R₁<R₂<4×R₁. The operating channel 9 thus opens out into the distal face 11d by an ovoid section whose projected surface with respect to the axis of the hole 12 decreases from the distal face 11d of the support in the direction of the proximal face 11p of the support 11.

Advantageously, the ovoid section of the operating channel 9 in the distal face 11d of the support 11 occupies a surface greater than or equal to at least 50% of the surface of the distal face of the support. In other words, the ovoid section of the hole 12 in the distal face 11d of the support 11 occupies a surface greater than or equal to at least 50% of the surface of the distal face 11d of the support. Preferably, the ovoid section of the operating channel 9 in the distal face 11d of the support 11 occupies a surface comprised between 50% and 60% of the surface of the distal face of the support 11. In other words, the ovoid section of the hole 12 in the distal face 11d of the support 11 occupies a surface comprised between 50% and 60% of the surface of the distal face of the support 11.

According to another aspect of the disclosure, the ovoid section of the operating channel 9 evolves from the distal face of the support 11, ending with a circular section. In the exemplary embodiment illustrated in the drawings, the ovoid section of the hole 12 evolves from the distal face of the support 11, ending with a circular section at the abutment face 13. It should be noted that the profile of the section of the hole 12 evolves by considering that the section of the hole 12 at the distal face 11d is greater than the section of the hole 12 at the abutment surface 13.

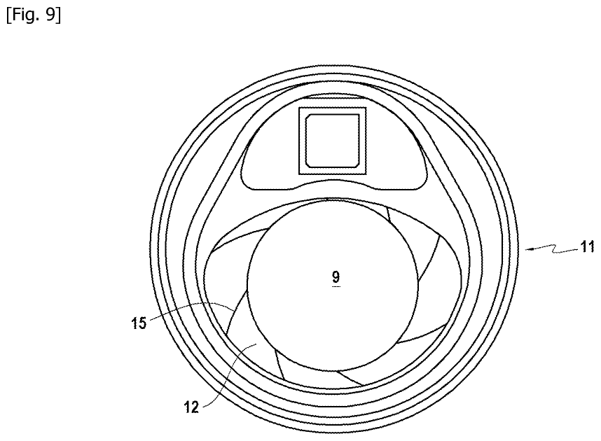

According to one advantageous alternative embodiment, the operating channel 9 is delimited by an inner surface provided, from the distal face 11d of the support 11, with helically-shaped grooves or ribs 15. In the exemplary embodiment illustrated in FIG. 9, the hole 12 is delimited by an inner surface provided, from the distal face 11d of the support 11, with helically-shaped grooves or ribs 15. The ribs or helices 15 are at least 3 in number, and will generate a vortex effect, aimed at mixing and rotating the fluids and mucus of different viscosities or shapes. This phenomenon aims to drive them into a helical movement in order to improve their evacuation in the operating channel 9.

According to another advantageous embodiment characteristic, the support 11 includes a passage 16 for mounting, as a video system V, an optical lens 17 or a vision sensor and at least one and, in the example illustrated, two light sources 18. The passage 16 is arranged in the support 11 to pass right therethrough by opening out onto the proximal face 11p and onto the distal face 11d. This passage 16 is independent with respect to the operating channel 9 arranged in the support 11. As shown in the drawings, the passage 16 opens out into the part of the distal face 11d, located opposite to the semi-circular edge 11₁, between the central edge 11₅ of the support 11 and the central edge 12₃ delimiting the hole 12. According to one advantageous characteristic, the optical lens or the vision sensor 17 and the light sources 18 are coated, along the distal face of the support, with a transparent protective material 19.

Advantageously, the optical lens 17 or the vision sensor and the light sources 18 are embedded into the mounting passage 16, by the transparent protective material 19.

Preferably, the support 11 is made of a hydrophobic material or coated with a hydrophobic coating.

The endoscope according to the disclosure has the advantage of including a distal head with an operating channel 9 having, at the distal face 11d, a widened section with respect to the operating channel of the previous endoscopes making it possible to reduce the suction phenomena. The widening of the operating channel 9 at the distal face also allows partially positioning tools or holding cumbersome bodies which will be evacuated during the withdrawal of the endoscope.

The disclosure is not limited to the examples described and represented because various modifications can be made thereto without departing from its scope.

1-12. (canceled)

13. A distal endoscope head including a support for a vision system, the support being delimited on one side, by a proximal face and, on the opposite side, by a distal face parallel to the proximal face and into which the vision system opens out, the support being traversed by at least one hole of an operating channel opening out into the distal face by an ovoid section whose projected surface with respect to the axis of the hole decreases from the distal face of the support in the direction of the proximal face of the support, the ovoid section of the hole of the operating channel in the distal face occupying a surface greater than or equal to at least 50% of the surface of the distal face of the support.

14. The distal head of claim 13, wherein the ovoid section of the hole of the operating channel in the distal face of the support occupies a surface comprised between 50% and 60% of the surface of the distal face of the support.

15. The distal head of claim 13, wherein the ovoid section of the hole of the operating channel evolves from the distal face of the support, ending with a circular section.

16. The distal head of claim 13, wherein the ovoid section of the hole of the operating channel evolves from the distal face of the support, ending with a circular section, at an abutment surface for one end of the conduit delimiting the operating channel.

17. The distal head of claim 13, wherein the ovoid section of the hole of the operating channel is delimited at the distal face, by a peripheral circular arc-shaped edge concentric with the outer surface of the support and connected on either side, by connecting edges, to a central circular arc-shaped edge whose radius is greater than the radius of the peripheral edge.

18. The distal head of claim 13, wherein the support is in the form of a ring delimited on one side by the distal face and on the other side by the proximal face, the distal and proximal faces being parallel to each other and perpendicular to the longitudinal axis of the ring.

19. The distal head of claim 13, wherein the hole of the operating channel is delimited by an inner surface provided, from the distal face of the support, with helically-shaped grooves or ribs.

20. The distal head of claim 13, wherein the support includes a passage for mounting, as a video system, an optical lens or a vision sensor and at least one light source, coated along the distal face of the support with a transparent protective material.

21. The distal head of claim 13, wherein the optical lens or the vision sensor and at least the light source are embedded into the mounting passage arranged in the support, by the transparent protective material.

22. The distal head of claim 13, wherein the support includes, from the proximal face, a cylindrical section extended by a narrowed section decreasing up to the distal face of the support to present at the distal face, a pseudo-triangular section.

23. The distal head of claim 20 or 21, wherein the passage opens out into the part of the distal face, located opposite to the semi-circular edge, between the central edge of the support and the central edge delimiting the hole.

24. An endoscope including an insertion tube connected, at its proximal part, to a control handle and equipped, at its distal part, with a distal head according to claim 13.
